(19) United States
(12) Patent Application Publication
(10) Pub. No.: US 2002/0077697 A1
(43) Pub. Date: Jun. 20, 2002

(54) Processed ratite carotid arteries as xenogeneic small bore vascular grafts

(76) Inventors: John Paul Ranieri, Austin, TX (US); Diane L. Hern-Anderson, Redwood City, CA (US); Anne Marie J. Gonin, Austin, TX (US); Brian Keith McIlroy, Georgetown, TX (US)

Correspondence Address: Timothy L. Scott, Senior Patent Counsel, SULZER MEDICA USA INC., 3 East Greenway Plaza, Suite 1600, Houston, TX, 77046, US

(21) Appl. No.: 09/737,505

(22) Filed: Dec. 15, 2000

(51) Int. Cl. A61F 2/06

(52) U.S. Cl. 623/1.41; 600/36

(57) ABSTRACT

The present invention is directed to various products and processes comprising segments of vasculature from long-necked birds for use as vascular grafts. Also contemplated is a process for preparing this vasculature for use as a small-bore vascular graft. The isolated vasculature of the present invention is of sufficient length to be used in a variety of applications, and may be stored for extended lengths of time after isolation or processing before implantation.

### FIELD OF THE INVENTION

The present invention relates to the use of ratite vasculature as xenogeneic graft material. More specifically, the present invention relates to the use of ratite carotid arteries as xenogeneic small-bore vascular grafts. The present invention also relates to segments of ratite vasculature prepared to be biocompatible with humans and to function as heterologous vascular graft tissue, as well as a method of isolating and producing non-immunogenic segments of ratite vasculature.

### BACKGROUND OF THE INVENTION

Materials currently used as small-bore vascular grafts are limited to autologous or cryo-preserved (cadaverous) allogenic saphenous vein and autologous internal mammary artery, radial artery, and gastroepiptoic artery. Autologous blood vessels have proven to be the most durable and to have the lowest failure rate over an extended period of time. The use of autologous vessels, however, requires additional or extended surgery, and in many cases, an additional incision is required in order to remove the vessel. This harvesting surgery not only increases the total operating time, but can also lead to complications and discomfort. Furthermore, for various reasons some patients do not have autologous vessels suitable for harvesting. For example, in some patients, vessels may be unavailable due to previous surgery or the vessels may be too small or varicose.

In light of the lack of availability or suitability of autologous vascular tissue in some instances, and the need for additional or extended surgery when autologous vessels are used, alternative graft materials that may be manufactured or harvested to be compatibly implanted as small-bore vascular grafts are continually sought. Alternative human tissues that have been evaluated include autologous pericardium, and allogenic human umbilical cord tissue. Synthetic materials that have been evaluated include polyethyleneterephthalate (PET), expanded polytetrafluoroethylene (EPTFE) grafts, and polyurethane grafts. Animal tissues from various species that have been studied include bovine, porcine, murine, canine, primate, ovine, caprine (goat) and ratite tissues.

One primary reason xenogeneic grafts from mammals fail when implanted into humans is that the vascular endothelium of mammalian tissues expresses the α-galactosamine α-Gal) epitope, which is a primary target for human antibodies. Cooper et al. (1996). Attempts to circumvent this problem have been explored. Cooper et al. (1996). They include evaluation of the suitability of ratites as organ and vascular tissue donors because ratite vascular endothelium does not express the α-Gal epitope. Taniguchi et al. (1996). Previous investigators have concluded, however, that because of other distinct anatomical, histologic, physiologic and immunologic species-related differences, ratite tissues are not likely to prove more useful as grafts or organs in humans than tissues from other mammalian species, despite their lack of α-Gal epitopes. Taniguchi et al. (1996).

To achieve acceptable performance and long-term patency rates in any graft material, the following characteristics are desired: hemocompatibility, low immunogenicity, biostability, non-cytotoxicity compliance similar to that of native blood vessels, adequate burst and suture pull strength, low kinking radius and suturability. Furthermore, the length of the graft is especially important when it is intended for use as a small bore graft (a graft with an internal diameter (“ID”) of less than about 6 mm). When such small bore vascular grafts are used for either coronary bypass situations or peripheral applications, they are often needed in lengths exceeding 20 cm. Meeting this length requirement becomes difficult when manufacturing grafts of biological origin because 3-4 mm ID vessels from other mammals (i.e., bovine, porcine, and ovine) taper significantly as they approach and exceed 20 cm in length. In addition, the number of side branches of these vessels increases, making both dissection and branch tie-off more time consuming. Alternatively, to obtain biological grafts greater than 20 cm in length, two shorter vessels have been sutured together end-to-end. This method has several disadvantages of its own, however, including requiring additional processing time to sew the two vessels together, blood leakage at the anastomosis, and blood flow disturbances and thrombosis which could ultimately cause graft failure due to discontinuity in the lumenal surface at the anastomoses.

Accordingly, xenogeneic small-bore vascular grafts which possess optimal characteristics for performance, biocompatibility, and biostability and which are of acceptable length without excessive branching are continually sought.

### SUMMARY OF THE INVENTION

To overcome shortcomings in the prior art, the inventors of the presently disclosed material have surprisingly and advantageously developed a method of processing vasculature isolated from ratites so that the immune response in the host is minimized. Specifically, the present disclosure teaches the use of ratite carotid artery for use as small-bore vascular grafts and a process for preparing ratite carotid artery for use as a small-bore vascular graft. By using the disclosed process the resultant graft material displays suture properties similar to native blood vessels, a good elasticity, a low rate of immune response in the host, low thrombogenicity and it may be stored for extended periods before implantation. A particular advantage of the ratite carotid artery vasculature disclosed in the present invention is that it may be isolated in lengths greater than 20 cm, making it convenient for use in a variety of applications, including coronary artery by-pass grafting; use as xenogeneic carotid artery; in peripheral grafting, especially radial grafting and peripheral grafting below the knee and across the knee; for intra-cranial artery bypass; and for shunts, including arterio-venous shunts.

Specifically contemplated to be within the scope of the present invention is a xenogeneic graft comprising a segment of non-human vasculature, in which the vasculature is isolated from the donor and is processed to be substantially biocompatible with humans. To promote biocompatibility, the vasculature is substantially decellularized by, for example, cellular lysis or lipid extraction. Decellularization may be achieved by treatment of the vasculature with various decellularizing agents including proteolytic enzymes such as ficin. Alternatively, decellularization may be achieved by storing the tissue in high salt high sugar (“HSHS”) or other hyperosmotic solution for a period of time, such as for about two weeks or up to about two months or more.

In another embodiment, the vasculature of the present invention is further treated to facilitate crosslinking of the collagen. Many chemical crosslinking agents are known to those of skill in the art, and virtually any agent that promotes crosslinking of the collagen, or any combination of agents that have the effect of promoting crosslinking, is contemplated to work according to the presently disclosed invention. Such agents, either alone or in combination, include the following: polyepoxy compounds such as polyethylene glycol diglycidal ether, glycerol polyglycidal ether, polyglycerol polyglycidal ether, sorbitol polyglycidal ether, or other polyglycidal ethers; carbodiimides such as cyanimide and 1-ethyl-3 (-3 dimethyl aminopropyl) carbodiimide hydrochloride (EDC); aldehydes and dialdehydes such as formaldehyde, glutaraldehyde, and dialdehyde starch; azides such as diphenylphosphorylazide (DPPA); genipin or other iridoid glycosides; bis-imidoesters; bis-N-succinimidyl derivatives; bifunctional aryl halides; bifunctional acylating agents such as hexamethylene-diisocyanate; bifunctional aryl halides; diketones; chloroformates such as ethylchloroformate and p-nitrophenyl chloroformate; or photosensitive crosslinkers containing one photoreactive group and one amine reactive group. Photoreactive chemical groups in these photosensitive crosslinkers include amylazides, diazoacetyl compounds, and azidophenyl derivatives. As stated, all of these substances are useful for crosslinking vasculature graft material, and are specifically contemplated to be useful, either alone or in combination, to crosslink collagen of the vasculature according to the present disclosure. Alternatively, any substance that functions to crosslink vascular collagen may be used according to the presently disclosed subject matter.

In another preferred embodiment, crosslinking of the collagen may be facilitated by a process such as photo-oxidation using a photo-catalyst and a dye. In such an embodiment, preferred photocatalyst and dye combinations include methylene blue, methylene green, rose bengal, riboflavin, proflavin, fluorescein, rosin, and pyridoxal-5-phosphate. In the alternative, virtually any catalyst that will cause transfer of hydrogen atoms or electrons upon activation is useful according to the present invention.

In yet another preferred embodiment, the vascular grafts of the present invention may further be modified with any agent, or combination of agents, that prevents clotting or thrombosis in or around the graft or otherwise promotes hemocompatibility of the graft. For example, any hemocompatibility-promoting agent may be covalently crosslinked to the vasculature. Exemplary hemocompatible agents that may be crosslinked to the vasculature include the enzymes streptokinase, urokinase, tissue plasminogen activator, tissue factor pathway inhibitor, thrombomodulin and Protein C; polyethylene glycol; phosphoryl choline; various alternative forms of heparin such as heparin sodium, low molecular weight heparin, heparin calcium, and synthetic heparin analogs; hirudin and hirulog; and prostaglandins, including stable prostaglandin analogs such as Iloprost.

In still another preferred embodiment, to facilitate the covalent crosslinking of the hemocompatible agent, crosslinking agents may again be employed. Crosslinking agents specifically contemplated to facilitate such crosslinking include all those listed above as useful to facilitate collagen crosslinking including traditional chemical agents as listed above, photooxidation methods and photocatalysts and dyes useful in the photooxidation methods as outlined above.

In another preferred embodiment, during the covalent crosslinking of the hemocompatible agent, the chemically activated vasculature is further contacted with an agent that promotes the formation of a stable intermediate, thereby prolonging the time or period over which the hemocompatible agent may crosslink to the vasculature. Agents useful for this purpose include n-hydroxysuccinimide (“NHS”) or n-hydroxysuccinimide sulfate (“NHSS”).

In any of the various preferred embodiments as outlined herein, the isolated vasculature is of variable lengths ranging from about 0.5 cm to about 50 cm or even to about 60 cm. Preferred lengths for the vascular grafts described herein include lengths equal to or greater than approximately 5 cm in length. More preferred are segments of vasculature greater than or equal to approximately 10 cm in length. Still more preferred are segments equal to or greater than approximately 15 cm in length or even equal to or greater than approximately 20 cm or 25 cm, or even 30, 35, or 40 cm in length. As will be readily understood by those skilled in the art, the presently disclosed grafts may be isolated and cut to any length useful for the individual practitioner's needs and the need of the recipient. Specifically contemplated are grafts isolated and processed according to the presently disclosed subject matter, of any length between about 0.5 cm and about 60 cm.

In preferred embodiments, the isolated vasculature has an internal diameter of less than about 8 mm, and a diameter less than about 6 mm in more preferred embodiments.

In another preferred embodiment, the vasculature of the present invention is a segment of carotid artery isolated from a donor ratite. Specific ratites that are contemplated to serve as useful donors of vasculature include the ostrich and emu. Additionally, while it is preferred that the donor be an ostrich, an emu or a similar ratite, it is specifically contemplated that any other long-necked bird capable of donating suitable vasculature may be the source of the xenogeneic grafts used in the present invention. Examples of long-necked birds that may be used include flamingos and other members of the family phoenicopteridae; herons or other ardidae; geese, swans or other anatidae; pelicans or other pelecanidae; or vultures or other cathartidae.

In various embodiments, the segments of vasculature isolated and processed according to the present disclosure will prove useful in a wide variety of grafting applications. Specific applications in which the segments should prove particularly useful include coronary artery bypass grafting; carotid artery grafting; peripheral vasculature grafting including grafting below or across the knee or radial grafting; intra-cranial artery bypass grafting; and as a shunt, such as an arterio-venous shunt.

In another preferred embodiment, the segment or segments of vasculature disclosed herein are aptly described as sterilized, isolated, substantially decellularized, crosslinked segment or segments of ratite vasculature comprising a hemocompatible agent. Such segments may be used, in various embodiments, as xenogeneic grafts in various species, including in virtually any mammal. In a particularly preferred embodiment, the segment or segments of ratite vasculature are used as xenogeneic grafts in humans.

In addition to the segments of ratite vasculature isolated and treated as described, the inventors of the presently disclosed subject matter expressly contemplate as within the scope of the present invention, methods by which the vasculature is processed to render it hemocompatible, biocompatible and non-immunogenic. Specifically, in one embodiment, the present invention provides a method of producing a xenogeneic graft comprising isolating a segment of vasculature from a ratite; substantially decellularizing the segment; contacting the segment with a crosslinking agent; and contacting the segment with a hemocompatible agent. In another preferred embodiment, the presently disclosed method applies to a segment of ratite vasculature that has been previously isolated, or isolated in a manner not in accordance with the present invention, but is then subsequently subjected to the process disclosed herein: the segment is substantially decellularized, contacted with a crosslinking agent; and contacted with a hemocompatible agent.

In various preferred embodiments, these methods further comprise contacting the vasculature with an additional agent to facilitate covalent crosslinking of the hemocompatible agent. In still other preferred embodiments, the covalent crosslinking of hemocompatible agent is carried out in the presence of an agent that extends the period of time over which the crosslinking occurs, by facilitating the formation of stable intermediates. Preferred crosslinking agents, preferred hemocompatible agents, and preferred stabilizing agents for use with the disclosed methods are the same as those outlined herein for the disclosed segments of vasculature.

In still other preferred embodiments, the methods disclosed herein may be applied to any length of vasculature that may be isolated, or may be specifically cut, as dictated by the particular characteristics of the donor animal, or as needed to satisfy the requirements of the individual practitioner or the needs of the recipient. Such lengths are preferably between 0.5 cm and 60 cm.

As with the aspects of the invention directed to the segments of vasculature, it is expressly contemplated that the methods disclosed herein may be applied to any vasculature isolated from a long-necked bird, with a ratite being the preferred donor. In a preferred embodiment, the methods are applied to isolated ratite carotid artery. In still other embodiments, the presently disclosed methods are preferably applied to carotid artery from an ostrich or emu. In a particularly preferred embodiment, the presently disclosed methods are applied to ostrich carotid artery.

In another preferred embodiment, the presently disclosed methods are applied to segments of ratite vasculature intended for use in various grafting applications in various species. Specifically, the method may be applied to segments intended for use as coronary artery bypass grafts; carotid artery grafting; peripheral applications such as below the knee and across the knee, or radial grafting; intra-cranial artery grafting; and as a shunt, including as an arterio-venous shunt. Species in which the grafts may advantageously be used include any mammal. In preferred embodiments, the recipient species is human.

In another preferred embodiment, the present disclosure provides for a ratite xenogeneic graft material produced by the process comprising decellularizing the segment of vasculature; contacting the segment with a crosslinking agent; and contacting the segment with a hemocompatible agent. In certain embodiments, such a segment of ratite vasculature is isolated or cut in any length as dictated by the particular characteristics of the donor animal, or as needed to satisfy the requirements of the individual practitioner or the needs of the recipient. Lengths specifically contemplated to be within the scope of the present disclosure include any length between about 0.5 cm. and about 60 cm.

In other preferred embodiments, the segments or segments of vasculature isolated and/or treated according to the presently disclosed process are isolated from any long-necked bird such as a ratite, including the ratites ostrich and emu. The ostrich is the preferred ratite donor. In further preferred embodiments, the ratite vasculature that is isolated and/or treated according to the presently disclosed processes is ratite carotid artery, with more preferred vasculature being ostrich carotid artery.

In yet another preferred embodiment, the ratite vasculature isolated and/or treated as described herein is used for coronary artery bypass grafting, carotid artery grafting, peripheral vasculature grafting, and as a shunt. Such applications may be made in any mammal, and in a preferred embodiment, the ratite vasculature isolated and/or treated according to the presently disclosed subject matter is used as a graft in humans.

To fully describe the details of the invention, a detailed description follows.

### DETAILED DESCRIPTION OF THE INVENTION

The following words will have the following definitions when used herein:

“Hemocompatible agent” as used herein to mean any agent that promotes compatibility between the vasculature of the present invention and the blood or body fluids. Examples of such agents include anti-platelet, anti-thrombogenic, thrombolytic and anti-coagulant agents, or any agent that minimizes the immunogenicity of the xenogeneic graft material disclosed herein.

Any numerical values recited herein include all values from the lower value to the upper value in increments of one unit provided that there is a separation of at least two units between any lower value and any higher value. As an example, if it is stated that the amount or concentration of a component or value of a process variable such as, for example, osmolality, temperature, pressure, time, length of vasculature, internal diameter and the like, is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

This invention involves harvesting small bore vasculature (vasculature with an internal diameter of less than about 6 mm) from ostriches or other ratites and processing them for use as xenogeneic small bore vascular grafts. To avoid eliciting an immune response, the tissue is decellularized. The tissue may also be crosslinked, including crosslinking with a hemocompatible agent, to provide additional biostability, minimize hematological problems, and increase the likelihood of patent anastomoses, good elasticity, and other desirable characteristics that will increase biocompatibility and biostability.

The ratite vascular tissue isolated and treated according to the present disclosure displays suture properties similar to native blood vessels, and is amenable to storage for extended periods of time. It is contemplated that the presently disclosed products and processes are useful in coronary artery bypass graft applications, peripheral bypass applications such as below- and across-the knee grafts and radial grafts, carotid artery grafting, and as a shunt including as an arterio-venous shunt.

Decellularization

Decellularization of the isolated vasculature assists in rendering the tissue nonimmunogenic and therefore increases the likelihood of histocompatibility with the host. Decellularization has the effect of removing substantial amounts of immunoreactive parenchymatous tissue from the vasculature intended for use as a xenogeneic graft. The goal is to remove as much of the immunoreactive tissue as possible so that just the collagen and elastin “skeleton” of the vasculature remains.

Various methods are employed to decellularize vascular tissues. For example, the tissue may be decellularized through a process of cellular lysis, or through lipid extraction or through a combination of these or other decellularization techniques. Exemplary methods include the use of enzymes such as ficin (though this may contribute to early matrix degradation and thus create long term biostability problems); the use of hypotonic solutions such as water to burst the cells (though this may disrupt the collagen matrix as well); and soaking the tissue in a hyperosmotic environment to disrupt the cell membranes. An exemplary hyperosmotic environment useful to decellularize the vasculature includes a high salt high sugar solution.

When a high salt high sugar solution is used, for example, the osmolality of the storage medium is preferably higher than the osmolality of 3.0 M NaCl solution, e.g., 4500 mOsm, and is preferably higher than about 6000 mOsm. The upper limit of the osmolality is imposed by the practicalities of handling the solution, such as, for example, the increasing viscosity that results from high solute content. The osmolality is also limited by the ability of the solvent to hold solute, such as the point at which it is saturated. Both sugar and salt contribute to the osmolality of this medium (as compared to the ionic strength of the solution, which results from inclusion of the salt, which dissociates in water) and the relative contributions of salt and sugar to the osmolality of the solution are not as important to the function of the solution as the total osmolality.

Many salts are suitable for use in a HSHS storage medium, but those which will function as described above to penetrate a tissue sample to inhibit hydration of the proteinaceous material in its native configuration are specifically contemplated. Due to their low cost, high solubility, and ready availability, sodium chloride and potassium chloride are the preferred salts for use. However, many other such salts are readily known in the art and readily available, including, for instance, the ammonium, sodium, calcium, magnesium, manganese, and potassium salts of halides, nitrites, nitrates, phosphides, phosphates, sulfites, sulfates, and alkanoic acids such as propionates, acetates, and formates, and specifically, the aforementioned sodium and potassium chloride, magnesium chloride, and sodium nitrite. All such salts, as well as many which are not listed here may be employed. The preferred weight/volume concentration of salt in the HSHS medium is between about 11.0% to about 29%, with concentrations between about 11.7% and about 28.4% more preferred.

As a general guideline, the desired contribution of the osmolality of the storage medium of the present invention attributable to the salt component is achieved by inclusion of, in the case of NaCl, about 2.0 to about 5.0 M NaCl in the medium (one liter) per kilogram final volume of water, or about 11.7 to about 28.4% (W:V) concentration, with the preferred range being about 2.25-4.0 M. For salts such as calcium chloride, the desired contribution to the osmolality of the medium is achieved by inclusion of from about 1.3 to about 3.4 M CaCl₂ in the medium, and so on.

Similarly, many sugars can be used as the second component of the storage medium of the present invention. Again because of its low cost, high solubility, and ready availability, sucrose may be the most convenient sugar to use in the storage medium. However, other sugars such as glucose, fructose, mannose, galactose and any other monosaccharides, disaccharides such as maltose, cellobiose, and lactose, trisaccharides, or polysaccharides such as amylose or amylopectin, as well as sugar derivatives such as sorbitol (derived from glucose by reduction of the aldehyde group), mannitol, etc., glycosides such as methyl glucoside (derived from glucose by acid-catalyzed reaction of methanol with glucose), or proteoglycans will also function in substantially the same way to achieve the desired result of maintaining the proteineous materials of a tissue sample in their native state, and all such sugars are contemplated as being useful as the sugar component. Preferred weight/volume amounts of sugar in the HSHS medium range from about 25% to about 90%, with concentrations between about 30% and about 85% more preferred.

In the case of sucrose, the desired contribution to the osmolality of the storage medium of the present invention can generally be obtained by using a concentration of from about 30 to about 85% W:V for example, sucrose, with the preferred range being from about 30 to about 80% W:V concentration sucrose. In other words, the contribution of the sugar to the total osmolality of the storage medium of the present invention ranges from about 3400 mOsm upwardly to the saturation point of the solvent.

It will also be recognized that some salts cause a decrease in pH when dissolved. This lowering of pH can have detrimental effect(s) on the collagenous tissue to be crosslinked. Even so, the magnesium chloride/sucrose solution can be used to advantage by, for instance, neutralizing the acidity of the solution by addition of sufficient magnesium hydroxide to raise the pH or by using a stronger butter. Such adjustments in pH are known to those skilled in the art and the resulting solution gives satisfactory results.

The storage medium is buffered to physiological pH by use of any of a number of commonly used buffers, one of the most commonly available being phosphate buffered saline (PBS), and that buffer is used to advantage in the medium of the present invention. Other suitable buffers include those containing potassium or sodium phosphate, or potassium or sodium chloride, such as a Good's buffer, e.g., HEPES, TES, or BES (Research Organics, Inc.), preferably at concentrations of from about 0.2 to about 1.0 M. However, the molar concentration of the buffer is not as important as the concentration of the other two components of the solution. Almost any concentration of buffer components which will maintain pH between about 3.5 and about 10, and preferably at about physiological pH, will function effectively in connection with the solution.

For a more detailed description of decellularization of collagenous tissues in high salt high sugar solutions, see U.S. patent application Ser. No. 08/435,867, the contents of which is hereby incorporated herein in its entirety.

Crosslinking

Steps may be taken to crosslink the collagen of heterologous graft tissue to further reduce the immune response and provide additional biostability in the host. Crosslinking may be achieved by any form of covalent crosslinking that increases the resistance of the tissue to enzymatic degradation. Traditional forms of crosslinking tissues include methods utilizing the following: polyepoxy compounds such as polyethylene glycol diglycidal ether, glycerol polyglycidal ether, polyglycerol polyglycidal ether, sorbitol polyglycidal ether, or other polyglycidal ethers; carbodiimides such as cyanimide and 1-ethyl-3(-3 dimethyl aminopropyl) carbodiimide hydrochloride (EDC); aldehydes and dialdehydes such as formaldehyde, glutaraldehyde, and dialdehyde starch; azides such as diphenylphosphorylazide (DPPA); bis-imidoesters; bis-N-succinimidyl derivatives; bifunctional aryl halides; bifunctional acylating agents such as hexamethylene-diisocyanate; bifunctional aryl halides; diketones; chloroformates such as ethylchloroformate and p-nitrophenyl chloroformate; or photosensitive crosslinkers containing one photoreactive group and one amine reactive group. Photoreactive chemical groups include arylazides, diazoacetyl compounds, and azidophenyl derivatives. All of these substances are useful for crosslinking vascular graft material, and are specifically contemplated to be useful with the ratite vasculature according to the present disclosure.

Alternatively, photooxidation methods for crosslinking may be used. Crosslinking by photooxidation advantageously results in crosslinked materials that maintain physical properties of untreated native tissues. Moore, et.al., (1994). Photooxidation methods are described in U.S. Pat. No. 5,332,475, specifically incorporated herein by reference in its entirety. This method may be applied to crosslink proteinaceous material such as the decellularized ratite vasculature used herein. The process comprises (1) soaking or “preconditioning” the material to be cross-linked in an aqueous buffered solution with a high osmolality, (2) incubating the material in an aqueous solution including sufficient photooxidative catalyst to catalyze the formation of inter- and intra-molecular cross-links by oxidation of the material, and (3) irradiating the material and the catalyst. All three steps of the process may be carried out by simply adding a photo catalyst to the soaking aqueous media and then subjecting the solution and tissue to irradiation. Alternatively, and in many cases preferably, the incubation and irradiation step are carried out in a different media than the initial preconditioning media.

Suitable solutions for the aqueous buffered media include water or low ionic strength buffers, phosphate buffered saline, high ionic strength buffers (those with μ=1.75 to 3.0), and organic buffers containing potassium or sodium phosphate, or potassium or sodium chloride. If the media used for the preconditioning step is different from that used for the incubating and irradiating step, the soaking media preferably has a high osmolality. In such a case, preferred solutions include sodium potassium, sodium chloride, sodium phosphate, potassium chloride, potassium phosphate, or Good's buffers. It may further prove useful to add sucrose or fructose to the preconditioning solutions.

The preconditioning solution will preferably have an osmolality of between about 390 mOsm to about 800 mOsm. When a second media is used for the incubation and irradiation step, it is preferable for the second solution to be a buffered solution with a lower osmolality. The osmolality is preferably between about 150 mOsm to about 400 mOsm, with an osmolality of about 290 to about 310 mOsm being more preferred.

Whether in a separate solution than the preconditioning step or not, the solution of the irradiation step also includes a catalyst and a dye. The vasculature being crosslinked is incubated in this solution until the ratio of the concentration of the medium to that of the material to be crosslinked is approximately at an equilibrium, or more specifically, in the range from about 10:1 to about 30:1. The length of time it takes to reach equilibrium depends upon various factors including the temperature, the pH and the osmolality of the solution or solutions in which the vasculature is being soaked and/or incubated and irradiated.

Once such a ratio is achieved, or whenever desired if an equilibrium is not deemed critical, the vasculature is irradiated to achieve photooxidation via the photocatalyst. Preferred photocatalysts include methylene blue, methylene green, rose bengal, riboflavin, proflavin, fluorescein, rosin, and pyridoxal-5-phosphate. Virtually any catalyst that will cause transfer of hydrogen atoms or electrons upon activation is useful. Sufficient concentration of the catalyst in the incubation/irradiation solution will generally be in ranges of about 0.0001% to about 0.25% (wt/vol). Preferred concentrations range from about 0.001% to about 0.1%, or any concentration sufficient to insure penetration into the material to be crosslinked and to catalyze the photooxidation of the protein.

Because this process for crosslinking is oxidative, it requires a sufficient amount of oxygen in the solution. Preferred amounts of oxygen in the solution include from about 5% to about 20%. As will be readily evident to one skilled in the art, the amount of oxygen in solution is maintained in various ways as may be necessary, depending upon the temperature of solution during irradiation. Methods for increasing the amount of oxygen in solution include bubbling air into the solution, agitating the solution, or any other means known in the art.

Temperature ranges in which the photooxidation process will be effective may range between those temperatures at which the tissue or solution freeze, and those temperatures at which the protein of the tissue being crosslinked denatures. Such temperatures are generally between about −2° C. to about 40° C., and are more preferably between about 0° C. and about 25° C.

Irradiation of the tissue in the catalyst and dye-containing solution is effective to achieve oxidation so long as the irradiating light is of a wavelength absorbed by the catalyst. Generally, this is achievable using incandescent, white or fluorescent light.

Tissues that have been successfully crosslinked via the photooxidation process exhibit excellent healing response in the host, are largely non-immunogenic and non-thrombogenic, and have high patency rates upon implantation. Anderson, J. M. et al., Fifth World Biomat. Congress, (1996), and Mclroy, B. K., et. al., 23rd Annual Mtg of Society for Biomaterials, (1997). They also display a desirable resistance to in vivo degradation and calcification.

Crosslinking with Hemocompatible Agents

Substances that may be crosslinked with the collagen structure include heparin, hirudin, and any other hemocompatible agent. For example, Hern et. al., showed that heparin can effectively be bound to collagen-based materials using a covalent cross-linker. Additional agents that may be additionally crosslinked include the enzymes streptokinase, urokinase, tissue plasminogen activator, tissue factor pathway inhibitor, thrombomodulin and protein C; polyethylene glycol; phosphoryl choline; various alternative forms of heparin such as heparin sodium, low molecular weight heparin, heparin calcium, and synthetic heparin analogs; hirudin and hirulog; and prostaglandins, including stable prostaglandin analogs such as Iloprost When additional substances such as heparin are to be crosslinked, the graft tissue may be advantageously pre-treated with an agent that will increase available binding sites on the tissue surface. Acceptable agents for this pre-treatment include various aminating agents such as hydroxyl amine sulfate. In general, preferred results are obtained by crosslinking the desired agents after photooxidation has taken place.

To assist in crosslinking antithrombogenic agents, the same crosslinking agents outlined above regarding collagen crosslinking are useful. Particularly useful agents include crosslinkers with an amine or carboxyl reactive group. It may further be preferable to add a substance such as n-hydroxysuccinimide (“NHS”) or n-hydroxysuccinimide sulfate (“NHSS”) to aid in the formation of a more stable intermediate, so that the reaction time is extended, thereby insuring more complete crosslinking of the heparin to the vasculature being treated.

Additional Treatments to Promote Compatibility and Storability

The decellularized, and optionally crosslinked material may be further treated to further facilitate biocompatibility and biostability, or the recipient of the graft may be treated to inhibit rejection of the xenograft. For example, it may be desirable to cross-link hydrophilic or hemocompatible polymers that allow the xenograft to resist protein degradation or platelet adhesion. Exemplary polymers imparting these characteristics include polyethylene glycol (“PEG”).

Additionally or alternatively, the graft lumen may be seeded or embedded with autologous endothelial cells and/or the outer surface of the graft may be seeded or embedded with myofibroblasts, fibroblasts, or smooth muscle cells. Any of these cell types may be genetically modified to secrete agents to enhance graft hemocompatibility.

A further additional or alternative process comprises treating the graft to promote in vivo re-endothelialization of the graft. For example, the graft may be treated so as to incorporate growth factors such as vascular endothelial growth factor (“VEGF”) or specific endothelial cell adhesion proteins or peptide sequences.

Pharmaceutical therapies may also be used to improve the long-term patency of grafts. For example, anti-platelet agents such as aspirin, ticlopidine hydrochloride, dipyridamole., calcium channel blockers, B-adrenergic blockers, and anagrelide may be administered to inhibit platelet aggregation at the site of the graft. These and other pharmaceutical therapies may be delivered via virtually any useful route of administration such as orally, parenterally, or even, for example, via a controlled release delivery system coupled with the graft. Such a system may involve, for example, an external sleeve positioned around the graft to deliver hemocompatible agents.

The grafts must generally also be sterilized prior to storage and/or implantation in the host. Sterilization may advantageously be achieved by dehydrating the treated vasculature with glycerol, then sterilizing it with ethylene oxide gas. When this process is utilized, the graft material must be rehydrated by soaking in saline solution or heparinized saline solution prior to implantation. Alternatively, the vasculature may be sterilized without being dehydrated by treating it with an iodine or peracetic acid solution, or any other solution-based chemical sterilants, or by exposing it to gamma irradiation.

Implantation

Once the vasculature has been decellularized, or both decellularized and otherwise treated to promote bio-compatibility and bio-stability such as by crosslinking of collagen or treatment with a hemocompatibility agent, it is preferably rinsed with a biocompatible solution, then is largely suitable for implantation.

### EXAMPLES

The following examples are illustrative only and are not intended to limit the scope of the invention. These methods have been shown by the inventors to be effective means of isolating and treating ratite vasculature to be useful as small bore xenogeneic graft material.

Materials and methods:

For the heparin cross-linking phase: active agents were Heparin formulation HE150 (Spectrum); 2-[N-morpholino]ethanesulfonic acid (“MES”) from Sigma, formulation M5287 at pH 3.5; 1-(3-Dimethylaminopropyl)-3-ethylcarbodimide hydrochloride (“EDC”) from Aldrich, formulation 16,146-2; N-Hydroxysuccinimide (“NHS”) from Sigma. formulation H7377; phosphate buffered saline (“PBS”) at pH 7; and Milli-Q water with a resistivity greater than 17.8 MΩ cm.

The MES Buffer comprises 24.4 g/500 ml of Milli-Q water. The heparin solution includes 50 mg/ml of heparin in MES buffer solution (1×). The EDC solution includes 25 mg/ml EDC in MES buffer solution (1×) and the NHS comprises 75 mg/ml NHS in MES buffer solution (1×). The PBS solution is composed of Milli-Q water, 0.137 M NaCl, 2.68 mM KCl, 8.10 mM Na2HPO4.7H20, and 2.57 mM KH2PO4 (anhydride).

### Example 1

### Isolation and Photo-Oxidation Ostrich Carotid Artery

Ostrich carotid artery was isolated, freed of surrounding tissue and side branches were tied off. The isolated arteries were stored in hypertonic solution (HSHS: high salt, high sugar) for 14 to 90 days. After storage in the hypertonic solution, the arteries were transferred to a 0.001% methylene blue/PBS (phosphate-buffered saline) solution and exposed to broad wavelength light for 44±14 hours to cross-link the tissue.

Following irradiation, the arteries were transferred to a 50% ethanol solution, for destaining, for 48 hours. The tissue was then stored in 50% ethanol or PBS. At this point, the tissue could either be sterilized (for subsequent storage and/or implantation), or could be further subjected to anti-thrombotic treatment.

### Example 2

### Anti-Thrombotic Treatment of Photo-Oxidized Ostrich Carotid Arteries

Subsequent to photo-oxidative crosslinking of the collagen skeleton of the isolated carotid artery, the artery was covalently crosslinked with heparin to further promote the anti-thrombogenic capacity of the resulting graft material. Materials used were as described above.

Anti-thrombogenic treatment began by removing the carotid grafts from the PBS solution. The grafts were then placed directly into separate glass tubes containing approximately 40 mls of 50 mg/ml heparin solution. Using a 3 ml syringe, heparin was injected into the center of each graft to displace the PBS.

The tubes were then placed on shaker for 2 hours at approximately 90 rpm, with the heparin solution completely covering the grafts. Approximately 5 minutes prior to removing the grafts from the heparin solution, an EDC:NHS 50:250 (molar) solution was prepared.

After two hours of heparin soaking, the heparin was discarded and replaced with the EDC:NHS solution. The EDC:NHS solution was placed in the glass vial containing the grafts and again a 3 ml syringe was used to inject the same solution inside the graft to displace any remaining heparin solution. The grafts were left in this solution overnight, at 4° C.

What is claimed is:

1. A xenogeneic graft comprising a segment of ratite carotid artery, wherein said artery is isolated from said ratite and is substantially decellularized.

2. A xenogeneic graft comprising a segment of ratite carotid artery, wherein said artery is: isolated from said ratite; and is substantially decellularized; and the collagen of said artery is at least partially crosslinked.

3. A xenogeneic graft comprising a segment of ratite carotid artery, wherein said artery: is isolated from said ratite; is substantially decellularized; has collagen that is at least partially cross-linked; and is contacted with a hemocompatible agent.

4. The graft of claim 1, 2 or 3, wherein said artery is further defined as having its lumen seeded with autologous endothelial cells; having its outer surface seeded with myofibroblasts, fibroblasts or smooth muscle cells; or as incorporating growth factors.

5. The graft of claim 1, 2 or 3, wherein said decellularization is achieved by treatment of the vasculature with a solution comprising at least about 50% high salt, high sugar solution or at least about 50% ethanol.

6. The graft of claim 1, 2, or 3, wherein said decellularization is achieved by cellular lysis or lipid extraction.

7. The graft of claim 2 or 3, wherein said crosslinking is promoted by diphenylphosphorylazide, 1-ethyl-3(-3dimethylaminopropyl)carbodiimide hydrochloride, or genepin.

8. The graft of claim 3, wherein said hemocompatible agent is selected from the group consisting of heparin, phosphorylcholine, and polyethylene glycol.

9. The graft of claim 3, wherein said artery is further contacted with 3-ethyl-3(3dimethyl-aminopropyl).

10. The graft of claim 1, 2, or 3, wherein said vasculature is at least about 5 cm in length.

11. The graft of claim 10, wherein said vasculature is at least about 10 cm in length.

12. The graft of claim 11, wherein said vasculature is at least about 20 cm in length.

13. The graft of claim 12, wherein said vasculature is at least about 40 cm in length.

14. The graft of claim 1, 2, or 3, wherein said ratite is ostrich.

15. The graft of claim 1, 2, or 3, for use in an application selected from the group consisting of coronary artery bypass grafting, carotid artery grafting, peripheral vasculature grafting, intra-cranial artery bypass and as an arterio-venous shunt.

16. A method of producing a xenogeneic vascular graft comprising substantially decellularizing an isolated segment of ratite carotid artery.

17. A method of producing a xenogeneic vascular graft comprising:
substantially decellularizing an isolated segment of ratite carotid artery; and
contacting said segment with an agent to promote crosslinking of collagen.

18. A method of producing a xenogeneic vascular graft comprising:
substantially decellularizing an isolated segment of ratite carotid artery;
contacting said segment with an agent to promote crosslinking of collagen; and
contacting said segment with a hemocompatible agent.

19. The method of claim 16, 17 or 18, wherein said decellularizing step comprises cellular lysis or lipid extraction.

20. The method of claim 16, 17 or 18, wherein said decellularizing step comprises soaking the tissue in a hyperosmotic solution comprising at least about 50% high salt, high sugar or at least about 50% ethanol.

21. The method of claim 17 or 18, wherein said crosslinking agent is 1-ethyl-3(3-dimethylaminopropyl)carbodiimide hydrochloride, diphenylphosphorylazide, or genepin.

22. The method of claim 17 or 18, wherein said crosslinking is achieved by a process comprising photooxidation of said vasculature while in the presence of a photooxidative catalyst.

23. The method of claim 18, wherein said hemocompatible agent is selected from the group consisting of heparin, phosphoryl choline, and polyethylene glycol.

24. The method of claim 16, 17 or 18, wherein said graft is at least 10 cm in length.

25. The method of claim 24, wherein said graft is at least 20 cm in length.

26. The method of claim 25, wherein said graft is at least 40 cm in length.

27. The method of claim 16, 17 or 18, wherein said ratite is an ostrich.

28. The method of claim 16, 17 or 18, wherein said graft is used in an application selected from the group consisting of coronary artery bypass grafting, carotid artery grafting, peripheral vasculature grafting, intra-cranial artery bypass and as an arteriovenous shunt.

29. A method of producing a xenogeneic graft comprising:
decellularizing an isolated segment of ratite vasculature by soaking said segment in a hyperosmotic solution comprising at least about 50% high salt, high sugar;
contacting said segment with 1-ethyl-3(-3dimethylaminopropyl)carbodiimide hydrochloride; and
contacting said segment with heparin.
